(19) United States
(12) Patent Application Publication
(10) Pub. No.: US 2017/0072005 A1
(43) Pub. Date: Mar. 16, 2017

(54) COMBINATIONS OF NMDAR MODULATING COMPOUNDS

(71) Applicant: NORTHWESTERN UNIVERSITY, Evanston, IL (US)

(72) Inventor: Joseph MOSKAL, Evanston, IL (US)

(21) Appl. No.: 15/309,390

(22) PCT Filed: May 6, 2015
(86) PCT No.: PCT/US2015/029477

§ 371 Date: Nov. 7, 2016

Related U.S. Application Data
(60) Provisional application No. 61/989,183, filed on May 6, 2014.

(51) Int. Cl. A61K 38/07; A61K 45/06

(52) U.S. Cl. CPC A61K 38/07; A61K 45/06

(57) ABSTRACT

This disclosure features combinations of NMDAR modulating compounds. This disclosure features combinations that include one or more NMDAR antagonists and GLYX-13 (each of which is sometimes referred to herein as a ‘component”). The beneficial effects of the combination are based, in part, on the finding that administration of GLYX-13 (e.g., a single dose) can reverse and/or prevent NMDAR antagonist-induced cognitive impairment (e.g., NMDAR antagonist-induced impairment in novel object recognition; e.g., induced through repeated dosing of the NMDAR antagonist).

### CROSS-REFERENCE TO RELATED APPLICATIONS

This application claims the benefit of U.S. Provisional Application No. 61/989,183, filed on May 6, 2014, which is incorporated herein by reference in its entirety.

### BACKGROUND OF THE INVENTION

An N-methyl-d-aspartate (NMDA) receptor (NMDAR) is a postsynaptic, ionotropic receptor that is responsive to, inter alia, the excitatory amino acids glutamate and glycine and the synthetic compound NMDA. The NMDA receptor controls the flow of both divalent and monovalent ions into the postsynaptic neural cell through a receptor associated channel (Foster et al., Nature 1987, 329:395-396; Mayer et al., Trends in Pharmacol. Sci. 1990, 11:254-260). The NMDA receptor has been implicated during development in specifying neuronal architecture and synaptic connectivity, and may be involved in experience-dependent synaptic modifications. In addition, NMDA receptors are also thought to be involved in long term potentiation and central nervous system disorders.

The NMDA receptor plays a major role in the synaptic plasticity that underlies many higher cognitive functions, such as memory acquisition, retention and learning, as well as in certain cognitive pathways and in the perception of pain (Collingridge et al., The NMDA Receptor, Oxford University Press, 1994). In addition, certain properties of NMDA receptors suggest that they may be involved in the information-processing in the brain that underlies consciousness itself.

The NMDA receptor has drawn particular interest since it appears to be involved in a broad spectrum of CNS disorders. For instance, during brain ischemia caused by stroke or traumatic injury, excessive amounts of the excitatory amino acid glutamate are released from damaged or oxygen deprived neurons. This excess glutamate binds to the NMDA receptors which opens their ligand-gated ion channels; in turn the calcium influx produces a high level of intracellular calcium which activates a biochemical cascade resulting in protein degradation and cell death. This phenomenon, known as excitotoxicity, is also thought to be responsible for the neurological damage associated with other disorders ranging from hypoglycemia and cardiac arrest to epilepsy. In addition, there are preliminary reports indicating similar involvement in the chronic neurodegeneration of Huntington's, Parkinson's, and Alzheimer's diseases. Activation of the NMDA receptor has been shown to be responsible for post-stroke convulsions, and, in certain models of epilepsy, activation of the NMDA receptor has been shown to be necessary for the generation of seizures. Neuropsychiatric involvement of the NMDA receptor has also been recognized since blockage of the NMDA receptor Ca⁺⁺ channel by the animal anesthetic PCP (phencyclidine) produces a psychotic state in humans similar to schizophrenia (reviewed in Johnson, K. and Jones, S., 1990). Further, NMDA receptors have also been implicated in certain types of spatial learning.

The NMDA receptor is believed to consist of several protein chains embedded in the postsynaptic membrane. The first two types of subunits discovered so far form a large extracellular region, which probably contains most of the allosteric binding sites, several transmembrane regions looped and folded so as to form a pore or channel, which is permeable to Ca⁺⁺, and a carboxyl terminal region. The opening and closing of the channel is regulated by the binding of various ligands to domains (allosteric sites) of the protein residing on the extracellular surface. The binding of the ligands is thought to affect a conformational change in the overall structure of the protein which is ultimately reflected in the channel opening, partially opening, partially closing, or closing.

NMDA receptor antagonists work to antagonize, or inhibit the action of, the N-Methyl-D-aspartate receptor (NMDAR). However, depressed NMDA receptor function can be associated with negative side effects, including those affecting cognitive ability.

Recently, an improved partial agonist of NMDAR, termed as GLYX-13, has been reported. GLYX-13 is exemplified by the following structure:

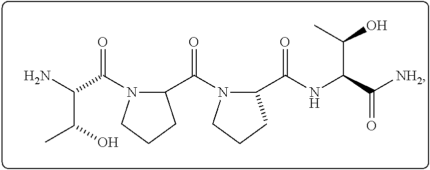

with a molecular weight: 413.47, and a chemical formula: C₁₈H₃₁N₅O₆. GLYX-13 exhibits nootropic, neuroprotective and antinociceptive activity, and enhances learning, memory and cognition in vivo.

### SUMMARY

This disclosure features combinations that include one or more NMDAR antagonists and GLYX-13 (each of which is sometimes referred to herein as a “component”). The beneficial effects of the combination are based, in part, on the finding that administration of GLYX-13 (e.g., a single dose) can reverse and/or prevent NMDAR antagonist-induced cognitive impairment (e.g., NMDAR antagonist-induced impairment in novel object recognition; e.g., induced through repeated dosing of the NMDAR antagonist). The combinations can further include one or more other biologically active ingredients (e.g., one or more other anti-depressant compounds) and/or one or more pharmaceutically acceptable excipients and/or carriers. The components of the combination (sometimes also referred to herein as chemical entities or chemical compounds) can be administered to a patient in a sequential manner (each component is administered at a different time) or in a substantially simultaneous manner. It will be appreciated that the components may be present in the same pharmaceutically acceptable carrier and, therefore, administered simultaneously. Alternatively, each of the components can be present in separate pharmaceutical carriers, such as, conventional oral dosage forms, or parenteral forms, (or one component may be oral and the other parenteral) that can be administered either simultaneously or sequentially. In some embodiments, pre-treatment with GLYX-13 (i.e., given prior to the administration of one or more NMDAR antagonists) can be particularly beneficial.

Accordingly, in one aspect, methods of substantially reversing or preventing cognitive impairment in a patient acutely administered a NMDAR antagonist are provided, which include administering an effective amount of GLYX-13.

In another aspect, methods of treating a cognitive impairment disorder in a patient in need thereof are provided, which include administering an effective amount of GLYX-13 and one or more NMDAR antagonists. The cognitive impairment disorder can be due to one or more of: deficit in cognitive ability, congenital defect, environmental factor(s), or drug induced and include, but are not limited to, learning disorders and/or dyslexia. In some embodiments, administering the effective amount of GLYX-13 occurs before or after the one or more NMDAR antagonists were acutely administered. In other embodiments, administering the effective amount of GLYX-13 occurs substantially simultaneously with acute administration of the one or more NMDAR antagonists.

In a further aspect, methods of treating a disorder, condition, or disease including, but not limited to: neurological or other disorders (e.g., stroke, psychotic disorder, pain (neuropathic pain), depression (major depression), Parkinson's disease, and Alzheimer's' disease); a central nervous system disease (e.g., neurodegenerative disease, stroke, traumatic brain injury, and spinal cord injury); schizophrenia; and/or depression (e.g., refractory depression), are provided, which include administering an effective amount of GLYX-13 and one or more NMDAR antagonists. In some embodiments, the GLYX-13 and the one or more NMDAR antagonists are administered substantially simultaneously. In other embodiments, the GLYX-13 and the one or more NMDAR antagonists are administered sequentially, e.g., the GLYX-13 is administered before or after the one or more NMDAR antagonists.

In one aspect, pharmaceutically acceptable compositions are provided, which include GLYX-13, one or more NMDAR antagonists, and one or more pharmaceutically acceptable excipients and/or carriers.

### BRIEF DESCRIPTION OF THE DRAWINGS

FIG. 1 is a diagram showing an overview of the novel object recognition model.

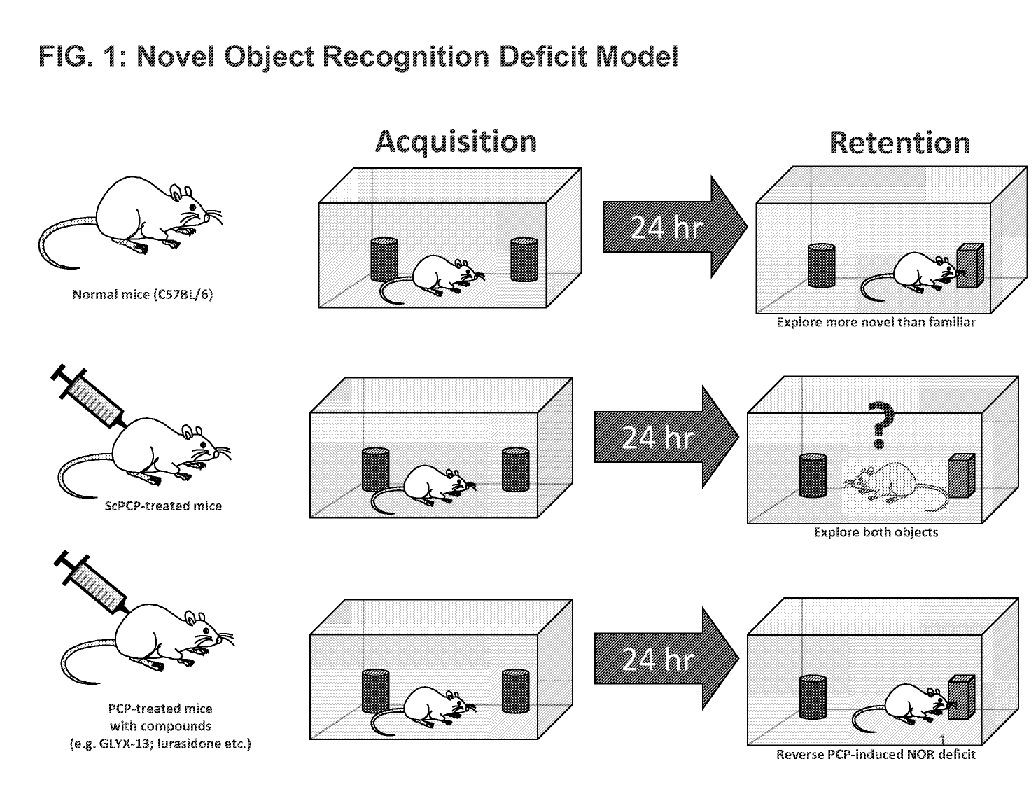

FIG. 2 shows mean±SEM Discrimination index score in the Novel object recognition test in adult male C57BL/6 male mice pre-treated with twice daily injection of ketamine (30 mg/kg IP) for 7 consecutive days followed by a sterile saline vehicle injection 1 hr before testing (ketamine group), GLYX-13 (1 mg/kg IV) injection 1 hr before testing (GLYX-13+ketamine group), or sterile saline injections twice daily for 7 days and a vehicle injection 1 hr before testing. The discrimination index is calculated using the following formula: (time spent exploring novel object−time spent exploring familiar object)/(total time spent in exploring both the novel and familiar objects). N=8-10 per group. * p<0.001, significant decrease in DI compared with the vehicle group, # p<0.001, significant reversal in DI compared with ketamine group (Fisher's PLSD post hoc test). The data in FIG. 2 demonstrates that GLYX-13 (1 mg/kg IV) reverses chronic ketamine-induced impairment in novel object recognition in mice.

FIG. 3 shows mean±SEM Discrimination index score in the Novel object recognition test in adult male C57BL/6 male mice pretreated with twice daily injection of PCP (10 mg/kg IP) for 7 consecutive days followed by a sterile saline vehicle injection 1 hr before testing (PCP group), GLYX-13 (1 mg/kg IV) injection 1 hr before testing (GLYX-13+PCP group), or sterile saline injections twice daily for 7 days and a vehicle injection 1 hr before testing. The discrimination index is calculated using the following formula: (time spent exploring novel object−time spent exploring familiar object)/(total time spent in exploring both the novel and familiar objects). N=8-10 per group. * p<0.001, significant decrease in DI compared with the vehicle group, # p<0.001, significant reversal in DI compared with PCP group (Fisher's PLSD post hoc test). The data in FIG. 3 demonstrates that GLYX-13 (1 mg/kg IV) reverses chronic phencyclidine-induced impairment in novel object recognition in mice.

FIG. 4 shows significant attenuation with pre-treatment of 3 mpk and 30 mpk of GLYX-13 in somatosensory cortex followed by ketamine.

FIG. 5 shows mean±SEM Discrimination index score in the Novel object recognition test in adult male C57BL/6 male mice pretreated with GLYX-13 (3 mg/kg iv) 30 min before ketamine (10 mg/kg sc) and tested 20 min later. The discrimination index is calculated using the following formula: (time spent exploring novel object−time spent exploring familiar object)/(total time spent in exploring both the novel and familiar objects). N=8-11 per group. *** p<0.0001, significant decrease in DI compared with the vehicle group, ## p<0.01, significant reversal in DI compared with ketamine group (Fisher's PLSD post hoc test). The data in FIG. 5 demonstrates that GLYX-13 (3 mg/kg iv) reverses acute ketamine (10 mg/kg sc) induced impairment in novel object recognition in mice.

FIG. 6 shows Mean (±SEM) number of stereotypy behaviour (circling and head weaving) in the open field in 2-3 month old male Sprague Dawley rats pretreated with GLYX-13 (3 mg/kg iv) 30 min before ketamine (10 mg/kg iv). Vehicle treated animals received saline vehicle injections instead of GLYX-13 and ketamine injections. Animals were placed into the open field immediately after the final dose, and behaviour was analysed for 20 min. N=8-12. The data in FIG. 6 demonstrates that GLYX-13 (3 mg/kg iv) inhibits ketamine (10 mg/kg iv) induced stereotypy in rats.

### DETAILED DESCRIPTION OF THE INVENTION

This disclosure features combinations that include one or more NMDAR antagonists and GLYX-13 (each of which is sometimes referred to herein as a “component”). The beneficial effects of the combination are based, in part, on the finding that administration of GLYX-13 (e.g., a single dose) can reverse and/or prevent NMDAR antagonist-induced cognitive impairment (e.g., NMDAR antagonist-induced impairment in novel object recognition; e.g., induced through repeated dosing of the NMDAR antagonist). The combinations can further include one or more other biologically active ingredients (e.g., one or more other anti-depressant compounds) and/or one or more pharmaceutically acceptable excipients and/or carriers. The components of the combination (sometimes also referred to herein as chemical entities or chemical compounds) can be administered to a patient in a sequential manner (each component is administered at a different time) or in a substantially simultaneous manner. It will be appreciated that the components may be present in the same pharmaceutically acceptable carrier and, therefore, administered simultaneously. Alternatively, each of the components can be present in separate pharmaceutical carriers, such as, conventional oral dosage forms, or parenteral forms, (or one component may be oral and the other parenteral) that can be administered either simultaneously or sequentially. In some embodiments, pre-treatment with GLYX-13 (i.e., given prior to the administration of one or more NMDAR antagonists) can be particularly beneficial.

“GLYX-13” is represented by the following formula:

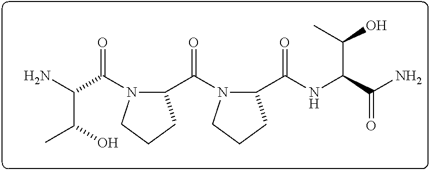

and includes polymorphs, hydrates, solvates, free bases, and/or suitable salt forms of the above compound.

“Treating” includes any effect, e.g., lessening, reducing, modulating, or eliminating, that results in the improvement of the condition, disease, disorder and the like.

The term “alkoxy” as used herein refers to a straight or branched alkyl group attached to an oxygen (alkyl-O—). Exemplary alkoxy groups include, but are not limited to, alkoxys of 1-6 or 2-6 carbon atoms, referred to herein as C₁-C₆ alkoxy, and C₂-C₆ alkoxy, respectively. Exemplary alkoxy groups include, but are not limited to methoxy, ethoxy, isopropoxy, etc.

The term “alkyl” as used herein refers to a saturated straight or branched hydrocarbon, such as a straight or branched group of 1-6, 1-4, or 1-3 carbon atoms, referred to herein as C₁-C₆alkyl, C₁-C₄alkyl, and C₁-C₃alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 3-methyl-2-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc. The term “haloalkyl” as used herein refers to a saturated straight or branched alkyl groups, in which one or more hydrogen atoms of the alkyl group are replaced with one or more independently selected halogens. The term “haloalkyl” encompasses alkyl groups in which all of hydrogen atoms of the alkyl group are replaced independently selected halogens (sometimes referred to as “perhalo” alkyl groups. Exemplary haloalkyl groups include, but are not limited to, CH₂F, CH₂CH₂C1, CF₃, CHFCH₂Cl.

The terms “halo” or “halogen” as used herein refer to F, Cl, Br, or I.

The term “oxo” as used herein refers to the radical ═O.

As used herein, the terms “NMDA receptor antagonist” and “NMDAR antagonist” generally both refer to a chemical entity that is capable of binding to a glycine binding site of an NMDA receptor rand works to antagonize, or inhibit, the action of the N-Methyl-D-aspartate receptor (NMDAR).

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The combinations described herein may also contain other active compounds providing supplemental, additional, or enhanced therapeutic functions.

The term “pharmaceutical composition” as used herein refers to a composition comprising at least one of the components of the combinations disclosed herein formulated together with one or more pharmaceutically acceptable carriers and/or excipients.

“Individual,” “patient,” or “subject” are used interchangeably and include any animal, including mammals, preferably mice, rats, other rodents, rabbits, dogs, cats, swine, cattle, sheep, horses, or primates, and most preferably humans. The combinations of the invention can be administered as described herein to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). In some embodiments, the mammal treated in the methods of the invention is a mammal in which treatment e.g., of pain or depression is desired.

The term “effective amount” refers to an amount of the subject component that will elicit the biological or medical response of a tissue, system, animal or human that is being sought by the researcher, veterinarian, medical doctor or other clinician. By way of example, an effective amount can be an amount effective to treat any of the diseases, disorders, and conditions described herein. Alternatively, an effective amount can refer the quantity needed to achieve a desired therapeutic and/or prophylactic effect, such as an amount of GLYX-13, which results reversing and/or preventing NMDAR antagonist-induced cognitive impairment (e.g., NMDAR antagonist-induced impairment in novel object recognition; e.g., induced through repeated dosing of the NMDAR antagonist).

The term “pharmaceutically acceptable salt(s)” as used herein refers to salts of acidic or basic groups that may be present in compounds used in the present combinations. Compounds included in the present combinations that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present combinations that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts. Compounds included in the present combinations that include a basic or acidic moiety may also form pharmaceutically acceptable salts with various amino acids. Compounds included in the present combinations may contain both acidic and basic groups; for example, one amino and one carboxylic acid group. In such a case, the compound can exist as an acid addition salt, a zwitterion, or a base salt.

The compounds included in the present combinations may contain one or more chiral centers and/or double bonds and, therefore, exist as geometric isomers, enantiomers or diastereomers. The enantiomer and diastereomers may be designated by the symbols “(+),” “(−).” “R” or “S,” depending on the configuration of substituents around the stereogenic carbon atom, but the skilled artisan will recognize that a structure may denote a chiral center implicitly. Geometric isomers, resulting from the arrangement of substituents around a carbon-carbon double bond or arrangement of substituents around a cycloalkyl or heterocyclic ring, can also exist in the compounds of the present invention. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Compounds included in the present combinations can exist in solvated as well as unsolvated forms with pharmaceutically acceptable solvents such as water, ethanol, and the like, and it is intended that the invention embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al, Nature Reviews Drug Discovery 2008, 7, 255). For example, if a compound of the invention or a pharmaceutically acceptable salt, hydrate or solvate of the compound contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

### Combination Components

GLYX-13 may be obtained by well-known recombinant or synthetic methods such as those described in U.S. Pat. Nos. 5,763,393 and 4,086,196 herein incorporated by reference. Also contemplated are polymorphs, hydrates, homologs, solvates, free bases, and/or suitable salt forms of GLYX 13 such as, but not limited to, the acetate salt. The peptide may be in cyclized or non-cyclized form as further described in U.S. Pat. No. 5,763,393. In some embodiments, a GLYX-13 analog may include an insertion or deletion of a moiety on one or more of the Thr or Pro groups such as a deletion of CH₂, OH, or NH₂ moiety. In other embodiments, GLYX-13 may be optionally substituted with one or more halogens, C₁-C₃ alkyl (optionally substituted with halogen or amino), hydroxyl, and/or amino Other compounds contemplated for use herein include Glycine-site partial agonists of the NMDAR disclosed in U.S. Pat. No. 5,763,393, U.S. Pat. No. 6,107,271, and Wood et al., Neuro. Report, 19, 1059-1061, 2008, the entire contents of which are herein incorporated by reference.

It may be understood that the peptides disclosed here can include both natural and unnatural amino acids, e.g., all natural amino acids (or derivatives thereof), all unnatural amino acids (or derivatives thereof), or a mixture of natural and unnatural amino acids. For example, one, two, three or more of the amino acids in GLYX-13 may each have, independently, a d- or l-configuration.

In some embodiments, the NMDAR antagonist is selected from the group consisting of ketamine, memantine, lanicemine (AZD6765), CERC-301, dextromethorphan, dextrorphan, phencyclidine, dizocilpine (MK-801), amantadine, ifenprodil, AV-101, AZD 6423, and riluzole, or a pharmaceutically acceptable salt or prodrug thereof. Also contemplated are derivatives of the aforementioned NMDAR antagonists.

In certain embodiments, the NMDAR antagonist has formula (I):

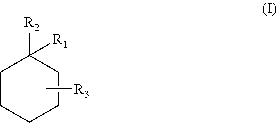

wherein:
R₁ is phenyl, thienyl, or benzothienyl, each of which is optionally substituted with from 1-3 substituents independently selected from the group consisting of halo; —OH; NR^{a}R^{b}, wherein each of R^{a} and R^{b} is independently selected from H and C₁-C₃ alkyl; C₁-C₃ alkyl; and C₁-C₃ alkoxy;
R₂ is —NR^{c}R^{d}, wherein each of R^{c} and R^{d} is independently selected from H and C₁-C₆ alkyl, which is optionally substituted with —OH or C₁-C₃ alkoxy; or R^{c} and R^{d} together with the nitrogen atom to which each is attached forms a 5-7 membered ring that is optionally substituted with from 1-2 independently selected C₁-C₃ alkyl; and
R₃ is H, oxo, or C₁-C₃ alkyl; or a pharmaceutically acceptable salt or prodrug thereof.

In certain embodiments, R₁ is phenyl, which is optionally substituted with from 1-3 substituents independently selected from the group consisting of halo; —OH; NR^{a}R^{b}, wherein each of R^{a} and R^{b} is independently selected from H and C₁-C₃ alkyl; C₁-C₃ alkyl; and C₁-C₃ alkoxy. For example, R₁ can be phenyl, 3-hydroxyphenyl, 3-methoxyphenyl, 3-aminophenyl, 3-methylphenyl, 4-fluorophenyl, 4-hydroxyphenyl, 3-methoxyphenyl, or 2-chlorophenyl. In other embodiments, R₁ is optionally substituted thienyl or optionally substituted benzothienyl.

In certain embodiments, R₂ is —NR^{e}R^{d}, wherein each of R^{e} and R^{d} is independently selected from H and C₁-C₆ alkyl, which is optionally substituted with —OH or C₁-C₃ alkoxy, e.g., H and C₁-C₆ alkyl, e.g., H and C₁-C₃ alkyl, e.g., one of R^{c} and R^{d} is H, and the other is and C₁-C₃ alkyl. For example, R₂ can be —NH(C₁-C₃ alkyl), such as —NH(CH₃). In other embodiments, R₂ is —NR^{e}R^{d}, wherein R^{c} and R^{d} together with the nitrogen atom to which each is attached forms a 5-7 membered ring that is optionally substituted with from 1-2 independently selected C₁-C₃ alkyl, such as piperidinyl.

In certain embodiments, R₃ is H or oxo.

In certain embodiments:

R₁ is phenyl, which is optionally substituted with from 1-3 substituents independently selected from the group consisting of halo; —OH; NR^{a}R^{b}, wherein each of Rₐ and R^{b} is independently selected from H and C₁-C₃ alkyl; C₁-C₃ alkyl; and C₁-C₃ alkoxy (e.g., R₁ is phenyl, 3-hydroxyphenyl, 3-methoxyphenyl, 3-aminophenyl, 3-methylphenyl, 4-fluorophenyl, 4-hydroxyphenyl, 3-methoxyphenyl, or 2-chlorophenyl);
R₂ is —NR^{c}R^{d}, wherein each of R^{c} and R^{d} is independently selected from H and C₁-C₆ alkyl, which is optionally substituted with —OH or C₁-C₃ alkoxy; e.g., H and C₁-C₆ alkyl, e.g., H and C₁-C₃ alkyl; e.g., one of R^{e} and R^{d} is H, and the other is and C₁-C₃ alkyl; e.g., R₂ can be —NH(C₁-C₃ alkyl), such as —NH(CH₃); and
R₃ is H or oxo (e.g., oxo).

In certain embodiments:

R₁ is phenyl, which is optionally substituted with from 1-3 substituents independently selected from the group consisting of halo; —OH; NR^{a}R^{b}, wherein each of R^{a} and R^{b} is independently selected from H and C₁-C₃ alkyl; C₁-C₃ alkyl; and C₁-C₃ alkoxy (e.g., R₁ is phenyl, 3-hydroxyphenyl, 3-methoxyphenyl, 3-aminophenyl, 3-methylphenyl, 4-fluorophenyl, 4-hydroxyphenyl, 3-methoxyphenyl, or 2-chlorophenyl);
R₂ is —NR^{c}R^{d}, wherein R^{c} and R^{d} together with the nitrogen atom to which each is attached forms a 5-7 membered ring that is optionally substituted with from 1-2 independently selected C₁-C₃ alkyl, such as piperidinyl; and

### R₃ is H or oxo (e.g., H).

In certain embodiments, R₁ is phenyl, R₂ is piperidinyl, and R₃ is H. For example, the compound can be phencyclidine.

In certain embodiments, R₁ is 2-chlorophenyl, R₂ is —NH(CH₃), and R₃ is oxo. For example, the compound can be ketamine, e.g., (S)-ketamine.

In certain embodiments, the NMDAR antagonist is memantine or amantadine. In certain embodiments, the NMDAR antagonist is dizocilpine (MK-801). In certain embodiments, the NMDAR antagonist is dextromethorphan or dextrorphan. In certain embodiments, the NMDAR antagonist is lanicemine (AZD6765), CERC-301, or ifenprodil. In certain embodiments, the NMDAR antagonist is AV-101 or AZD 6423.

In some embodiments, the NMDAR antagonist is selected from the group consisting of nitrous oxide, atomoxetine, dextrallorphan, diphenidine, eticyclidine, gacyclidine, ibogaine, methoxetamine, nitromemantine, rolicyclidine, tenocyclidine, methoxydine, tiletamine, neramexane, eliprodil, etoxadrol, dexoxadrol, methadone, WMS-2539, NEFA, remacemide, delucemine, 8A-PDHQ, aptiganel (Cerestat, CNS-1102), HU-211, remacemide, rhynchophylline, TK-40, Traxoprodil (CP-101,606), 1-Aminocyclopropanecarboxylic acid (ACPC), kynurenic acid or a derivative thereof, 2-carboxytetrahydroquinoline or a derivative thereof, 2-carboxyindole or a derivative thereof, 4-hydroxy-2-quinoline or a derivative thereof, 4-hydroxyquinoline or a derivative thereof, quinoxaline-2,3-dione or a derivative thereof, trycyclic antagonists, lacosamide, L-phenylalanine, midafotel, and aptiganel, or a pharmaceutically acceptable salt or prodrug thereof.

See also, e.g., those described in Kvist et. al., J. Biol. Chem. 2013 288: 33124-33135, which is incorporated herein by reference in its entirety. See also, e.g., those described in Traynelis et al., Pharmacological Reviews 2010, 62, 405, which is incorporated herein by reference in its entirety (e.g., CGP-61594; CGP-58411; ACEA-1011 and 1021; L-701,324; (R)-APS; (R)-AP7; PMPA; (R)-CPP; NVP-AAM077; PPDA; (R)-a-AA; PBPD; UBP141; CGS-19755 (selfotel); CGP-43487; CGP-40116; Conantokins, e.g., Br, G, Pr1, Pr2, Pr3, R, and T; radiprodil; and MK-0657).

In certain embodiments, the NMDAR antagonist is kynurenic acid or a derivative thereof, 2-carboxytetrahydroquinoline or a derivative thereof, 2-carboxyindole or a derivative thereof, 4-hydroxy-2-quinoline or a derivative thereof, 4-hydroxyquinoline or a derivative thereof, quinoxaline-2,3-dione or a derivative thereof, or a trycyclic antagonist. Examples of such compounds are described herein and, e.g., in Danysz et al., Pharmacological Reviews 1998, 50, 597, which is incorporated herein by reference in its entirety.

### Methods

In one aspect, methods of substantially reversing or preventing cognitive impairment in a patient acutely administered a NMDAR antagonist are provided, which include administering an effective amount of GLYX-13.

In another aspect, methods of treating a cognitive impairment disorder in a patient in need thereof are provided, which include administering an effective amount of GLYX-13 and one or more NMDAR antagonists. The cognitive impairment disorder can be due to one or more of: deficit in cognitive ability, congenital defect, environmental factor(s), or drug induced and include, but are not limited to, learning disorders and/or dyslexia. In some embodiments, the effective amount of GLYX-13 occurs before or after the one or more NMDAR antagonists were acutely administered. In other embodiments, the effective amount of GLYX-13 occurs substantially simultaneously with acute administration of the one or more NMDAR antagonists.

In a further aspect, methods of treating a disorder, condition, or disease including, but not limited to: neurological or other disorders (e.g., stroke, psychotic disorder, pain (e.g., neuropathic pain), depression (e.g., major depression), Parkinson's disease, and Alzheimer's' disease); a central nervous system disease (e.g., neurodegenerative disease, stroke, traumatic brain injury, and spinal cord injury); schizophrenia; and/or depression (e.g., refractory depression), are provided, which include administering the combinations described herein, e.g., an effective amount of GLYX-13 and one or more NMDAR antagonists. Other exemplary conditions include, but are not limited to, a learning disorder, autistic disorder, attention-deficit hyperactivity disorder, anxiety, migraine, Tourette's syndrome, phobia, post-traumatic stress disorder, dementia, memory deficits associated with aging, AIDS dementia, Huntington's disease, spasticity, myoclonus, muscle spasm, bipolar disorder, neuropathic pain, a substance abuse disorder, urinary incontinence, ischemia, special learning disorders, seizures, post-stroke convulsions, brain ischemia, hypoglycemia, cardiac arrest and epilepsy. In some embodiments, the GLYX-13 and the one or more NMDAR antagonists are administered substantially simultaneously. In other embodiments, the GLYX-13 and the one or more NMDAR antagonists are administered sequentially, e.g., the GLYX-13 is administered before or after the one or more NMDAR antagonists.

Contemplated methods include a method of treating autism and/or an autism spectrum disorder in a patient need thereof, which include administering the combinations described herein, e.g., an effective amount of GLYX-13 and one or more NMDAR antagonists. In an embodiment, a method for reducing the symptoms of autism in a patient in need thereof is contemplated, comprising administering the combinations described herein, e.g., an effective amount of GLYX-13 and one or more NMDAR antagonists. For example, upon administration, the combinations may decrease the incidence of one or more symptoms of autism such as eye contact avoidance, failure to socialize, attention deficit, poor mood, hyperactivity, abnormal sound sensitivity, inappropriate speech, disrupted sleep, and perseveration. Such decreased incidence may be measured relative to the incidence in the untreated individual or an untreated individual(s).

In some embodiments, patients suffering from autism also suffer from another medical condition, such as Fragile X syndrome, tuberous sclerosis, congenital rubella syndrome, and untreated phenylketonuria.

In some embodiments, methods of treating a disorder in a patient need thereof are contemplated, wherein the disorder is selected from group consisting of: cerebral ischemia, stroke, brain trauma, brain tumors, acute neuropathic pain, chronic neuropathic pain, sleep disorders, drug addiction, depression, certain vision disorders, ethanol withdrawal, anxiety, memory and learning disabilities, autism, epilepsy, AIDS dementia, multiple system atrophy, progressive supra-nuclear palsy, Friedrich's ataxia, Down's syndrome, fragile X syndrome, tuberous sclerosis, olivio-ponto-cerebellar atrophy, cerebral palsy, drug-induced optic neuritis, peripheral neuropathy, myelopathy, ischemic retinopathy, diabetic retinopathy, glaucoma, cardiac arrest, behavior disorders, impulse control disorders, Alzheimer's disease, memory loss that accompanies early stage Alzheimer's disease, attention deficit disorder, ADHD, schizophrenia, amelioration of opiate, nicotine addiction, ethanol addition, traumatic brain injury, spinal cord injury, post-traumatic stress syndrome, and Huntington's chorea that includes administering the combinations described herein, e.g., an effective amount of GLYX-13 and one or more NMDAR antagonists.

In some embodiments, contemplated herein are methods of treating attention deficit disorder, ADHD (attention deficit hyperactivity disorder), schizophrenia, anxiety, amelioration of opiate, nicotine and/or ethanol addiction (e.g., method of treating such addiction or ameliorating the side effects of withdrawing from such addiction), spinal cord injury diabetic retinopathy, traumatic brain injury, post-traumatic stress syndrome and/or Huntington's chorea, in a patient in need thereof, that includes administering the combinations described herein, e.g., an effective amount of GLYX-13 and one or more NMDAR antagonists. For example, patients suffering from schizophrenia, addiction (e.g. ethanol or opiate), autism, Huntington's chorea, traumatic brain injury, spinal cord injury, post-traumatic stress syndrome and diabetic retinopathy may all be suffering from altered NMDA receptor expression or functions.

For example, provided herein is a method of treating depression in a patient need thereof, comprising administering the combinations described herein, e.g., an effective amount of GLYX-13 and one or more NMDAR antagonists. In certain embodiments, the treatment-resistant patient is identified as one who has been treated with at least two types of antidepressant treatments prior to administration of the combinations described herein. In other embodiments, the treatment-resistant patient is one who is identified as unwilling or unable to tolerate a side effect of at least one type of antidepressant treatment.

The most common depression conditions include Major Depressive Disorder and Dysthymic Disorder. Other depression conditions develop under unique circumstances. Such depression conditions include but are not limited to Psychotic depression, Postpartum depression, Seasonal affective disorder (SAD), mood disorder, depressions caused by chronic medical conditions such as cancer or chronic pain, chemotherapy, chronic stress, post traumatic stress disorders, and Bipolar disorder (or manic depressive disorder).

Refractory depression occurs in patients suffering from depression who are resistant to standard pharmacological treatments, including tricyclic antidepressants, MAOIs, SSRIs, and double and triple uptake inhibitors and/or anxiolytic drugs, as well non-pharmacological treatments such as psychotherapy, electroconvulsive therapy, vagus nerve stimulation and/or transcranial magnetic stimulation. A treatment resistant-patient may be identified as one who fails to experience alleviation of one or more symptoms of depression (e.g., persistent anxious or sad feelings, feelings of helplessness, hopelessness, pessimism) despite undergoing one or more standard pharmacological or non-pharmacological treatment. In certain embodiments, a treatment-resistant patient is one who fails to experience alleviation of one or more symptoms of depression despite undergoing treatment with two different antidepressant drugs. In other embodiments, a treatment-resistant patient is one who fails to experience alleviation of one or more symptoms of depression despite undergoing treatment with four different antidepressant drugs. A treatment-resistant patient may also be identified as one who is unwilling or unable to tolerate the side effects of one or more standard pharmacological or non-pharmacological treatment.

In yet another aspect, a method for enhancing pain relief and for providing analgesia to an animal is provided. In some embodiments, methods are provided for treating neuropathic pain. The neuropathic pain may be acute or chronic. In some cases, the neuropathic pain may be associated with a condition such as herpes, HIV, traumatic nerve injury, stroke, post-ischemia, fibromyalgia, reflex sympathetic dystrophy, complex regional pain syndrome, spinal cord injury, sciatica, phantom limb pain, diabetic neuropathy, and cancer chemotherapeutic-induced neuropathic pain. Methods for enhancing pain relief and for providing analgesia to a patient are also contemplated.

In certain embodiments, methods for treating schizophrenia are provided. For example, paranoid type schizophrenia, disorganized type schizophrenia (i.e., hebephrenic schizophrenia), catatonic type schizophrenia, undifferentiated type schizophrenia, residual type schizophrenia, post-schizophrenic depression, and simple schizophrenia may be treated using the methods and compositions contemplated herein. Psychotic disorders such as schizoaffective disorders, delusional disorders, brief psychotic disorders, shared psychotic disorders, and psychotic disorders with delusions or hallucinations may also be treated using the compositions contemplated herein.

Paranoid schizophrenia may be characterized where delusions or auditory hallucinations are present, but thought disorder, disorganized behavior, or affective flattening are not. Delusions may be persecutory and/or grandiose, but in addition to these, other themes such as jealousy, religiosity, or somatization may also be present.

Disorganized type schizophrenia may be characterized where thought disorder and flat affect are present together.

Catatonic type schizophrenia may be characterized where the subject may be almost immobile or exhibit agitated, purposeless movement. Symptoms can include catatonic stupor and waxy flexibility.

Undifferentiated type schizophrenia may be characterized where psychotic symptoms are present but the criteria for paranoid, disorganized, or catatonic types have not been met.

Residual type schizophrenia may be characterized where positive symptoms are present at a low intensity only.

Post-schizophrenic depression may be characterized where a depressive episode arises in the aftermath of a schizophrenic illness where some low-level schizophrenic symptoms may still be present.

Simple schizophrenia may be characterized by insidious and progressive development of prominent negative symptoms with no history of psychotic episodes.

In some embodiments, methods are provided for treating psychotic symptoms that may be present in other mental disorders, including, but not limited to, bipolar disorder, borderline personality disorder, drug intoxication, and drug-induced psychosis.

In another embodiment, methods for treating delusions (e.g., “non-bizarre”) that may be present in, for example, delusional disorder are provided.

Also provided are methods for treating social withdrawal in conditions including, but not limited to, social anxiety disorder, avoidant personality disorder, and schizotypal personality disorder.

Additionally, methods are provided for treating obsessive-compulsive disorder (OCD).

Also provided herein is a method of modulating an autism target gene expression in a cell comprising contacting a cell with the combinations described herein, e.g., an effective amount of GLYX-13 and one or more NMDAR antagonists. The autism gene expression may be for example, selected from ABAT, APOE, CHRNA4, GABRAS, GFAP, GRIN2A, PDYN, and PENK. In another embodiment, a method of modulating synaptic plasticity in a patient suffering from a synaptic plasticity related disorder is provided, comprising administering the combinations described herein, e.g., an effective amount of GLYX-13 and one or more NMDAR antagonists.

In another embodiment, a method of treating Alzheimer's disease, or e.g., treatment of memory loss that e.g., accompanies early stage Alzheimer's disease, in a patient in need thereof is provided, comprising administering the combinations described herein, e.g., an effective amount of GLYX-13 and one or more NMDAR antagonists. Also provided herein is a method of modulating an Alzheimer's amyloid protein (e.g., beta amyloid peptide, e.g. the isoform Aβ₁₋₄₂), in-vitro or in-vivo (e.g. in a cell) comprising contacting the protein with the combinations described herein, e.g., an effective amount of GLYX-13 and one or more NMDAR antagonists. For example, in some embodiments, GLYX-13 or another disclosed compound may block the ability of such amyloid protein to inhibit long-term potentiation in hippocampal slices as well as apoptotic neuronal cell death. In some embodiments, a disclosed compound (e.g., GLYX-13) may provide neuroprotective properties to a Alzheimer's patient in need thereof, for example, may provide a therapeutic effect on later stage Alzheimer's—associated neuronal cell death.

In some embodiments, the patient is a human, e.g. a human pediatric patient.

The present disclosure contemplates “combination therapy,” which includes (but is not limited to) co-administering an effective amount of GLYX-13 and one or more NMDAR antagonists, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually days, weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection.

Combination therapy also can embrace the administration of the therapeutic agents as described above in further combination with other biologically active ingredients and non-drug therapies. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In some embodiments, one or more of the components of the combinations described herein may be administered parenterally to a patient including, but not limited to, subcutaneously and intravenously. In some embodiments, one or more of the components of the combinations described herein may also be administered via slow controlled i.v. infusion or by release from an implant device. In some embodiments, a patient has substantial improvement in, e.g., cognitive impairment, after 1 hour, 2 hours 4 hours, 8 hours, 12 hours, after 1 day, after 1 week, after 2 days, after 3 days, after 4 days, after 5 days, after 6 days, or even after 8 days of a one (single) dose administration of GLYX-13.

A therapeutically effective amount of a disclosed compound required for use in therapy varies with the nature of the autism condition being treated, the length of treatment time desired, the age and the condition of the patient, and is ultimately determined by the attending physician. In general, however, doses employed for adult human treatment typically are in the range of about 0.01 mg/kg to about 1000 mg/kg per day (e.g., about 0.01 mg/kg to about 100 mg/kg per day, about 0.01 mg/kg to about 10 mg/kg per day, about 0.1 mg/kg to about 100 mg/kg per day, about 0.1 mg/kg to about 50 mg/kg per day, about 0.1 mg/kg to about 10 mg/kg per day) of each component of the combinations described herein. In certain embodiments, doses of GLYX-13 employed for adult human treatment typically are in the range of about 0.01 mg/kg to about 100 mg/kg per day (e.g., about 0.01 mg/kg to about 10 mg/kg per day, about 0.1 mg/kg to about 100 mg/kg per day, about 0.1 mg/kg to about 50 mg/kg per day, about 0.1 mg/kg to about 10 mg/kg per day, about 1 mg/kg per day). In certain embodiments, doses of an NMDAR antagonist employed for adult human treatment typically are in the range of about 0.01 mg/kg to about 100 mg/kg per day (e.g., about 0.1 mg/kg to about 100 mg/kg per day, about 0.1 mg/kg to about 50 mg/kg per day, about 10 mg/kg per day or about 30 mg/kg per day). The desired dose may be conveniently administered in a single dose, or as multiple doses administered at appropriate intervals, for example as two, three, four or more sub-doses per day.

A number of factors may lead to each component of the combinations described herein being administered over a wide range of dosages. When given in combination with other therapeutic agents, the dosage of the compounds of the present invention may be given at relatively lower dosages. In certain embodiments, the dosage of GLYX-13 may be from about 1 ng/kg to about 100 mg/kg. The dosage of GLYX-13 may be at any dosage including, but not limited to, about 1 ug/kg, 25 ug/kg, 50 ug/kg, 75 ug/kg, 100 u ug/kg, 125 ug/kg, 150 ug/kg, 175 ug/kg, 200 ug/kg, 225 ug/kg, 250 ug/kg, 275 ug/kg, 300 ug/kg, 325 ug/kg, 350 ug/kg, 375 ug/kg, 400 ug/kg, 425 ug/kg, 450 ug/kg, 475 ug/kg, 500 ug/kg, 525 ug/kg, 550 ug/kg, 575 ug/kg, 600 ug/kg, 625 ug/kg, 650 ug/kg, 675 ug/kg, 700 ug/kg, 725 ug/kg, 750 ug/kg, 775 ug/kg, 800 ug/kg, 825 ug/kg, 850 ug/kg, 875 ug/kg, 900 ug/kg, 925 ug/kg, 950 ug/kg, 975 ug/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

In some embodiments, the disclosed compound, e.g. GLYX-13, may be dosed at amount that reverses or prevents cognitive impairment.

Disclosed compounds may be provided as part of a liquid or solid formulation, for example, aqueous or oily suspensions, solutions, emulsions, syrups, and/or elixirs. The compositions may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl hydroxybenzoate and sorbic acid. Contemplated compounds may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water (e.g., water for injection).

In some embodiments, disclosed compounds, e.g. GLYX-13, may be provided as part of an aqueous composition that is suitable for intravenous injection. In certain embodiments, such compositions can include: (i) 60 mg/mL to about 200 mg/mL (e.g., about 125 mg/mL to about 175 mg/mL; e.g., about 150 mg/mL or about 75 mg/mL) of a pharmaceutically active compound having the formula:

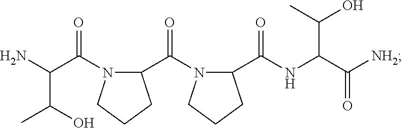 [SMILES: CC(O)C(N)C(=O)N1CCCC1C(=O)N1CCCC1C(=O)NC(C(N)=O)C(C)O]

or a pharmaceutically acceptable salt thereof; (ii) water (e.g., water for injection); and (iii) an acid; wherein the stable, aqueous composition has a pH of from about 3.9 to about 5.5 (e.g., from about 4.0 to about 5.0, from about 4.2 to about 5.0, from about 4.1 to about 4.7, from about 4.2 to about 4.8, about 4.0, about 4.5) at 25° C. In certain embodiments, such compositions can be disposed within a receptacle (e.g., a prefilled syringe or vial), in which the amount of the compound is extractable as at least one single dose. In certain embodiments, the single dose can have a volume of about 1 mL to about 4 mL (e.g., 3 mL).

In certain embodiments, the aqueous compositions can include about 200 mg to about 500 mg (e.g., about 450 mg; about 375; or about 225 mg) of the pharmaceutically active compound.

In certain embodiments, the acid can be selected from the group consisting of fumaric acid, malic acid, lactic acid, hydrochloric acid, hydrobromic acid, acetic acid, citric acid, phosphoric acid, nitric acid, sulfuric acid, and ascorbic acid. In certain embodiments, the acid provides chloride ions in the aqueous composition (e.g., hydrochloric acid).

In certain embodiments, upon administration of a dose of the aqueous liquid composition that includes about 150 mg/mL of the pharmaceutically active compound and has a volume of about 3 mL to a patient, a physiological osmolality of from about 800 mOsmol/kg to about 900 mOsmol/kg is obtained in said patient. In other embodiments, upon administration of a dose of the stable, aqueous liquid composition that includes about 75 mg/mL of the pharmaceutically active compound and has a volume of about 3 mL to a patient, a physiological osmolality of from about 375 mOsmol/kg to about 475 mOsmol/kg is obtained in said patient.

### Examples

Novel Object recognition Test (“NOR”) testing in mice was adapted from (Hashimoto K, Fujita Y, Shimizu E, Iyo M (2005). Phencyclidine-induced cognitive deficits in mice are improved by subsequent subchronic administration of clozapine, but not haloperidol. European journal of pharmacology 519(1-2): 114-117). See also, e.g., Rajagopal, et al., Current Pharmaceutical Design 2014, 20, 1. The NOR box is an open box made out of Plexiglas (52 cm L; 52 cm W; 31 cm H). The dimensions of the box we used for mice was identical to the one used for rats. The box was positioned approximately 30 cm above the floor. The walls of the box have white background, as opposed to the black background in the rat NOR studies. We found that C57BL/6 mice explored more in the white background when compared to the black one. Three days prior to testing, mice were habituated to the empty NOR arena for an hour. The NOR test in mice is similar to that previously employed to study rat NOR except that the acquisition and retention trials were 10 min in duration, followed by an intertrial interval (ITI) of 24 hours during which the mice were returned to their home cages, whereas in rats, the acquisition and retention trials were three min in duration separated by a one min ITI (Horiguchi M, Meltzer HY (2012). The role of 5-HT1A receptors in phencyclidine (PCP)-induced novel object recognition (NOR) deficit in rats. Psychopharmacology 221(2): 205-215). We compared 3, 5, and 10 min times for acquisition and retention trial explorations and found 10 minutes to be optimal for reliable data collection. Both trials were recorded for blind scoring later on.

All data are expressed as mean±S.E.M. Exploration data were analysed by a two-way analysis of variance (ANOVA). This detected the main effect of drug treatment, the main effect of task, and the interaction between drug treatment and object exploration. When a significant effect was found, further analysis by a post hoc Student's t-test was performed to compare the times spent exploring the novel and familiar object. The primary endpoint was the discrimination index (DI). The DI (novel-familiar/novel+familiar) data were analysed using one-way ANOVA followed by the Bonferroni test when a significant effect was detected by the ANOVA.

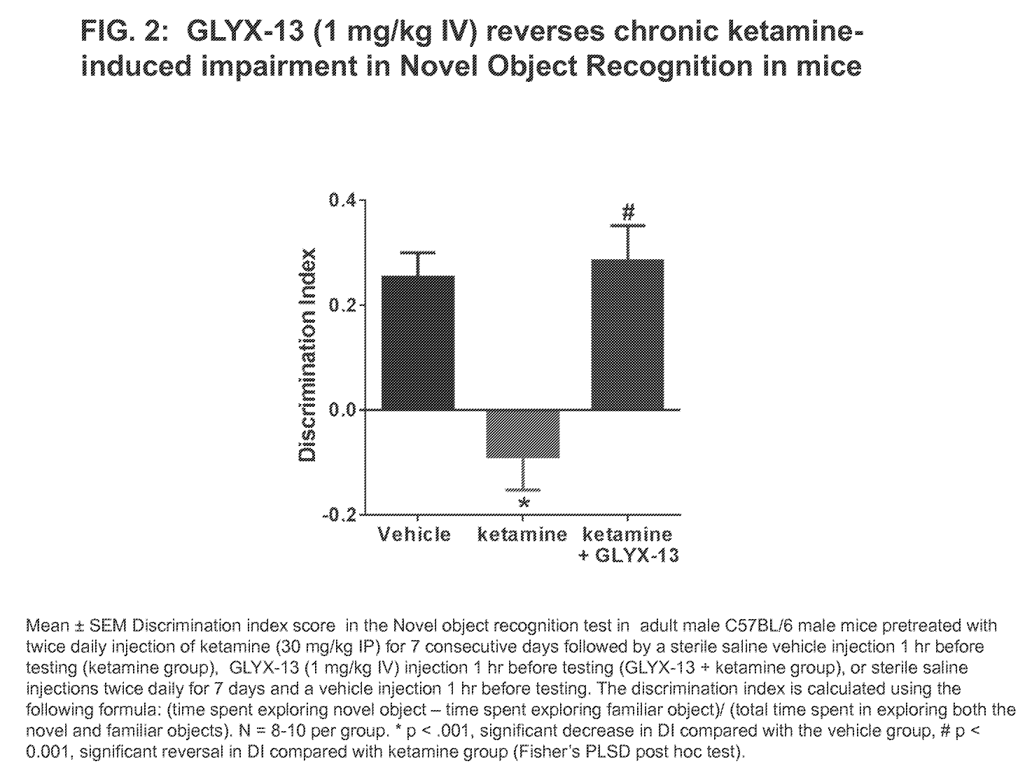

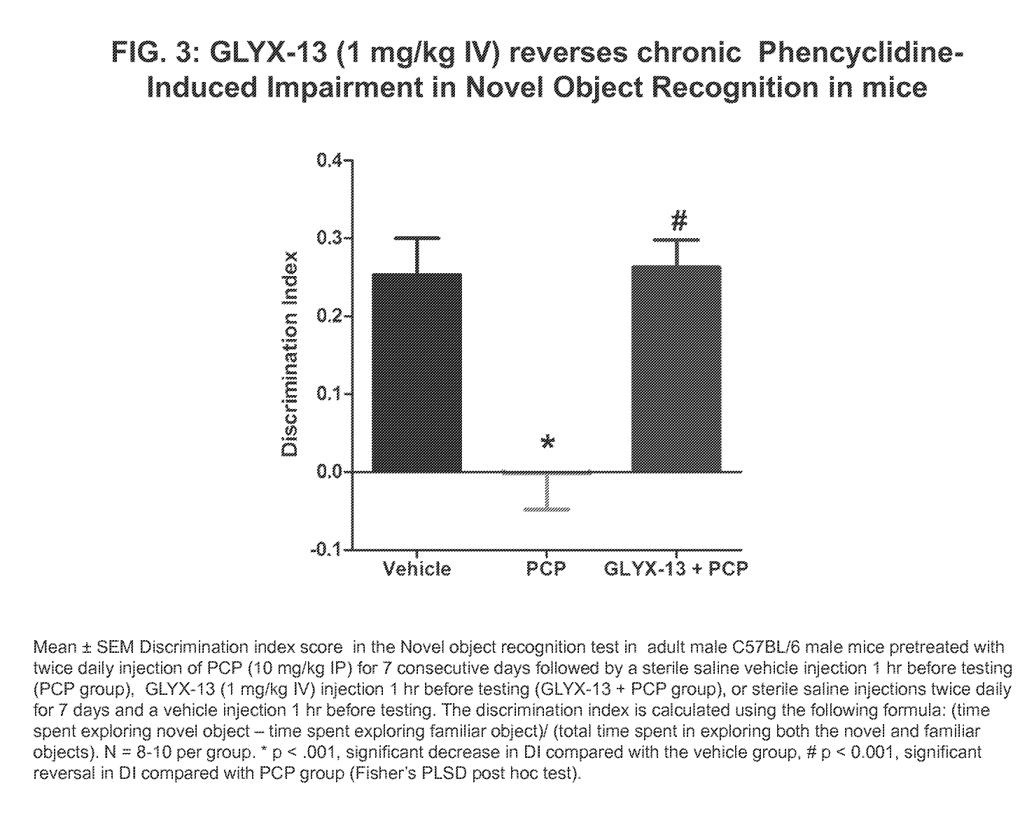

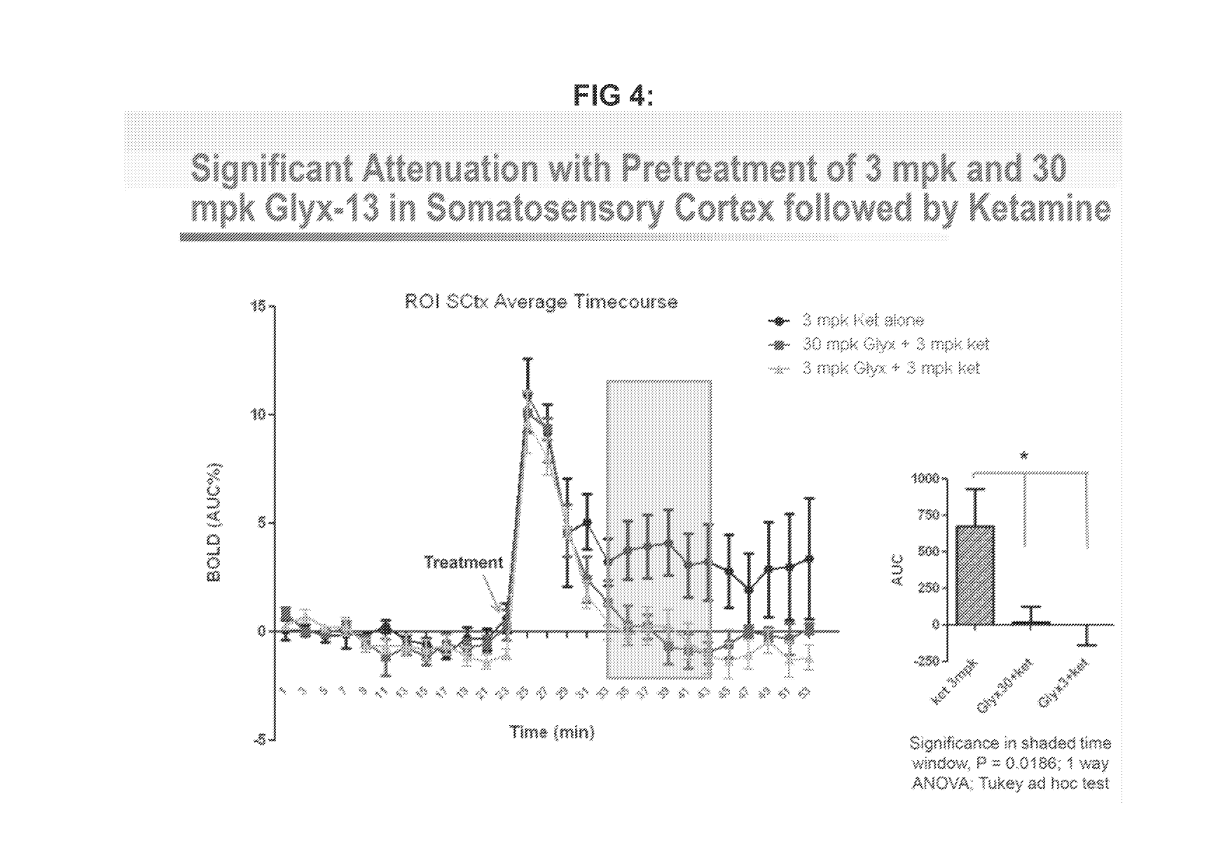

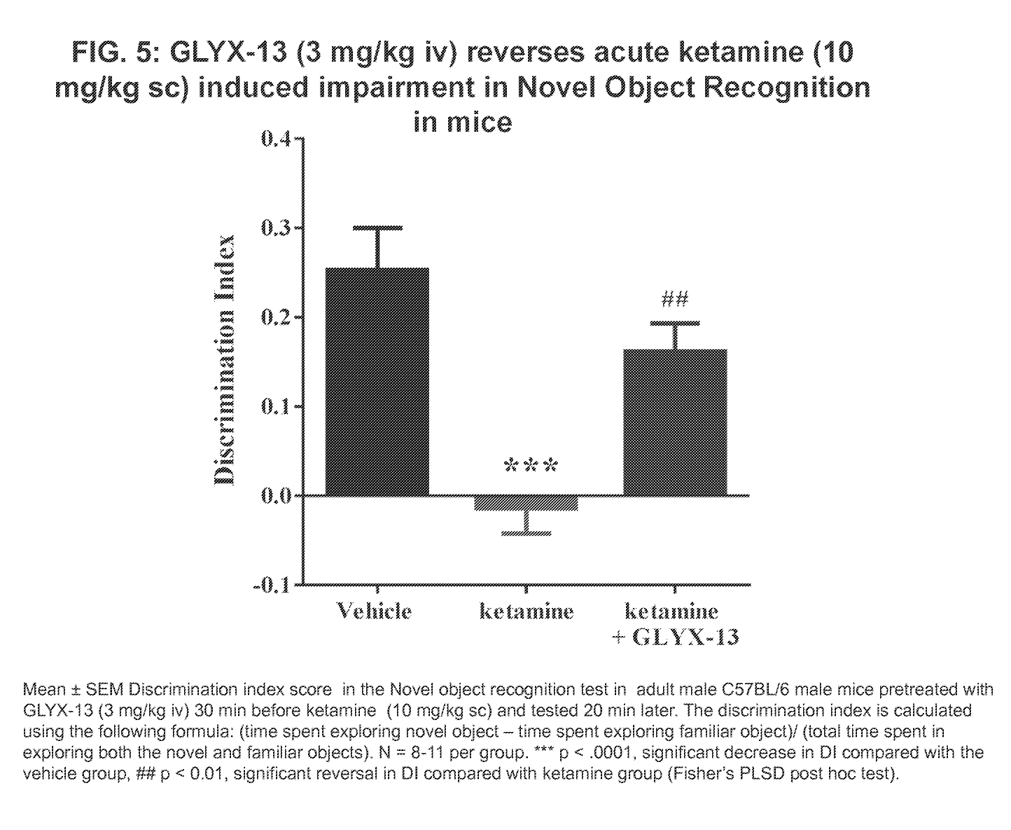

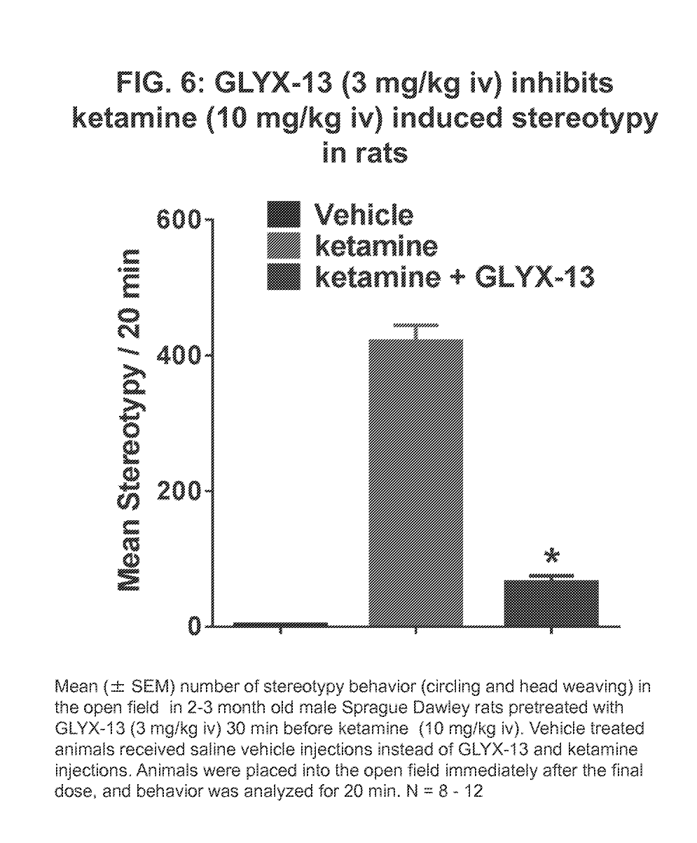

The data in FIG. 2 demonstrates that GLYX-13 (1 mg/kg IV) reverses chronic ketamine-induced impairment in novel object recognition in mice. The data in FIG. 3 demonstrates that GLYX-13 (1 mg/kg IV) reverses chronic phencyclidine-induced impairment in novel object recognition in mice. FIG. 4 shows significant attenuation with pre-treatment of 3 mpk and 30 mpk of GLYX-13 in somatosensory cortex followed by ketamine. The data in FIG. 5 demonstrates that GLYX-13 (3 mg/kg iv) pre-treatment reverses acute ketamine (10 mg/kg sc) induced impairment in novel object recognition in mice. The data in FIG. 6 demonstrates that GLYX-13 (3 mg/kg iv) inhibits ketamine (10 mg/kg iv) induced stereotypy in rats.

Those skilled in the art will recognize, or be able to ascertain using no more than routine experimentation, many equivalents to the specific embodiments of the invention described herein. Such equivalents are intended to be encompassed by the following claims.

The entire contents of all patents, published patent applications, websites, and other references cited herein are hereby expressly incorporated herein in their entireties by reference.

What is claimed is:

1. A method of substantially reversing or preventing cognitive impairment in a patient acutely administered a NMDAR antagonist, comprising administering an effective amount of GLYX-13.

2. The method of claim 1, wherein the administering an effective amount of GLYX-13 occurs before the NMDAR antagonist was acutely administered.

3. The method of claim 1, wherein the administering an effective amount of GLYX-13 occurs after the NMDAR antagonist was acutely administered.

4. The method of claim 1, wherein the administering an effective amount of GLYX-13 occurs substantially simultaneously with acute administration of the NMDAR antagonist.

5. A method of treating a cognitive impairment disorder in a patient in need thereof, comprising administering GLYX-13 and a NMDAR antagonist.

6. The method of claim 5, wherein the cognitive impairment disorder is due to one or more of: deficit in cognitive ability, congenital defect, environmental factor(s), or drug induced.

7. The method of claim 5, wherein the cognitive impairment disorder is a learning disorder and/or dyslexia.

8. A method of treating a neurological or other disorder comprising administering GLYX-13 and a NMDAR antagonist.

9. The method of claim 8, wherein the disorder is selected from the group consisting of: stroke, psychotic disorder, pain (neuropathic pain), depression (major depression), Parkinson's disease, and Alzheimer's' disease.

10. A method for treating a central nervous system disease in a patient in need thereof, comprising administering GLYX-13 and a NMDAR antagonist.

11. The method of claim 10, wherein the central nervous system disease is selected from the group consisting of: neurodegenerative disease, stroke, traumatic brain injury, and spinal cord injury.

12. A method of treating schizophrenia in a patient in need thereof, comprising administering GLYX-13 and a NMDAR antagonist.

13. A method of treating depression in a patient in need thereof, comprising administering GLYX-13 and a NMDAR antagonist.

14. The method of claim 13, wherein the depression is refractory depression.

15. The method of any one of claims 5-14, wherein the GLYX-13 and the NMDAR antagonist are administered substantially simultaneously.

16. The method of any one of claims 5-14, wherein the GLYX-13 and the NMDAR antagonist are administered sequentially.

17. The method of claim 16, wherein the GLYX-13 is administered before the NMDAR antagonist.

18. The method of claim 16, wherein the GLYX-13 is administered after the NMDAR antagonist.

19. A pharmaceutically acceptable composition comprising GLYX-13 and a NMDAR antagonist.

20. A method of any one of claims 1-18, or the pharmaceutical composition of claim 19, wherein the NMDAR antagonist has formula (I): 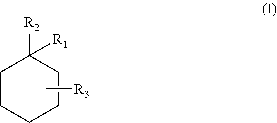
wherein:
R₁ is phenyl, thienyl, or benzothienyl, each of which is optionally substituted with from 1-3 substituents independently selected from the group consisting of halo; —OH; NR^{a}R^{b}, wherein each of R^{a} and R^{b} is independently selected from H and C₁-C₃ alkyl; C₁-C₃ alkyl; and C₁-C₃ alkoxy;
R₂ is —NR^{c}R^{d}, wherein each of R^{c} and R^{d} is independently selected from H and C₁-C₆ alkyl, which is optionally substituted with —OH or C₁-C₃ alkoxy; or R^{c} and R^{d} together with the nitrogen atom to which each is attached forms a 5-7 membered ring that is optionally substituted with from 1-2 independently selected C₁-C₃ alkyl; and
R₃ is H, oxo, or C₁-C₃ alkyl;
or a pharmaceutically acceptable salt or prodrug thereof.

21. The method of claim 20, wherein R₁ is phenyl, which is optionally substituted with from 1-3 substituents independently selected from the group consisting of halo; —OH; NR^{a}R^{b}, wherein each of R^{a} and R^{b} is independently selected from H and C₁-C₃ alkyl; C₁-C₃ alkyl; and C₁-C₃ alkoxy.

22. The method of claim 21, wherein R₁ is phenyl, 3-hydroxyphenyl, 3-methoxyphenyl, 3-aminophenyl, 3-methylphenyl, 4-fluorophenyl, 4-hydroxyphenyl, 3-methoxyphenyl, or 2-chlorophenyl.

23. The method of claim 20, wherein R₂ is —NH(C₁-C₃ alkyl) or piperidinyl.

24. The method of claim 20, wherein R₃ is H or oxo.

25. The method of claim 20, wherein R₁ is phenyl, R₂ is piperidinyl, and R₃ is H.

26. The method of claim 20, wherein R₁ is 2-chlorophenyl, R₂ is —NH(CH₃), and R₃ is oxo.

27. A method of any one of claims 1-18, or the pharmaceutical composition of claim 19, wherein the NMDAR antagonist is selected from the group consisting of ketamine, memantine, lanicemine (AZD6765), CERC-301, dextromethorphan, dextrorphan, phencyclidine, dizocilpine (MK-801), amantadine, ifenprodil, AV-101, AZD 6423, and riluzole, or a pharmaceutically acceptable salt or prodrug thereof.

28. The method of claim 27, wherein the NMDAR antagonist is ketamine.

29. The method of claim 28, wherein the NMDAR antagonist is (S)-ketamine.

30. The method of claim 27, wherein the NMDAR antagonist is phencyclidine.

31. The method of claim 27, wherein the NMDAR antagonist is memantine or amantadine.

32. The method of claim 27, wherein the NMDAR antagonist is dizocilpine (MK-801).

33. The method of claim 27, wherein the NMDAR antagonist is dextromethorphan or dextrorphan.

34. The method of claim 27, wherein the NMDAR antagonist is lanicemine (AZD6765), CERC-301, AV-101, AZD 6423, or ifenprodil.

35. A method of any one of claims 1-18, or the pharmaceutical composition of claim 19, wherein the NMDAR antagonist is selected from the group consisting of nitrous oxide, atomoxetine, dextrallorphan, diphenidine, eticyclidine, gacyclidine, ibogaine, methoxetamine, nitromemantine, rolicyclidine, tenocyclidine, methoxydine, tiletamine, neramexane, eliprodil, etoxadrol, dexoxadrol, methadone, WMS-2539, NEFA, remacemide, delucemine, 8A-PDHQ, aptiganel (Cerestat, CNS-1102), HU-211, remacemide, rhynchophylline, TK-40, Traxoprodil (CP-101,606), 1-Aminocyclopropanecarboxylic acid (ACPC), kynurenic acid or a derivative thereof, 2-carboxytetrahydroquinoline or a derivative thereof, 2-carboxyindole or a derivative thereof, 4-hydroxy-2-quinoline or a derivative thereof, 4-hydroxyquinoline or a derivative thereof, quinoxaline-2,3-dione or a derivative thereof, trycyclic antagonists, lacosamide, L-phenylalanine, midafotel, and aptiganel, or a pharmaceutically acceptable salt or prodrug thereof.

36. The method of claim 35, wherein the NMDAR antagonist is a 2-carboxy tetrahydroquinoline or a derivative thereof.

37. The method of claim 36, wherein the NMDAR antagonist is selected from the group consisting of: 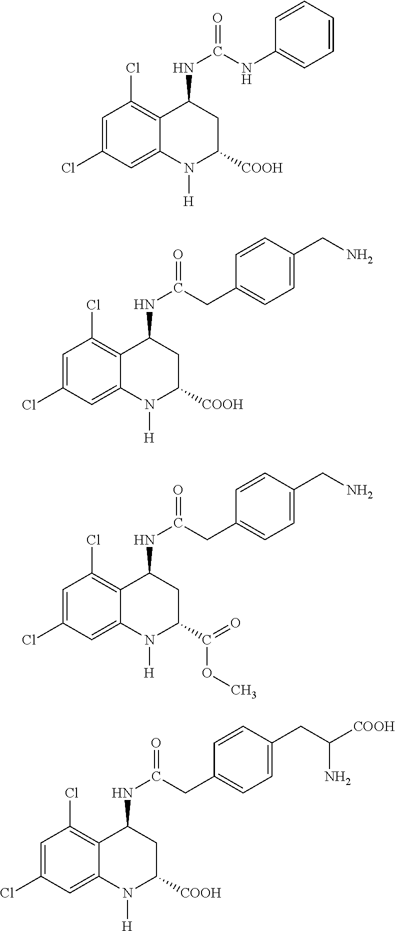
or a pharmaceutically acceptable salt or prodrug thereof.

38. The method of claim 35, wherein the NMDAR antagonist is a 2-carboxyindole or a derivative thereof.

39. The method of claim 38, wherein the NMDAR antagonist is selected from the group consisting of: 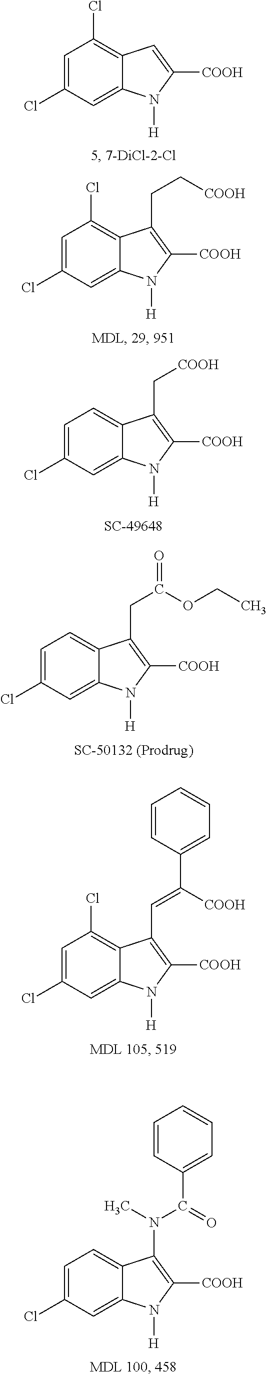 [SMILES: O=C(O)c1cc2c(Cl)cc(Cl)cc2[nH]1 ; O=C(O)CCc1c(C(=O)O)[nH]c2cc(Cl)cc(Cl)c12 ; O=C(O)Cc1c(C(=O)O)[nH]c2cc(Cl)ccc12 ; CCOC(=O)Cc1c(C(=O)O)[nH]c2cc(Cl)ccc12 ; O=C(O)/C(=C\c1c(C(=O)O)[nH]c2cc(Cl)cc(Cl)c12)c1ccccc1 ; CN(C(=O)c1ccccc1)c1c(C(=O)O)[nH]c2cc(Cl)ccc12] 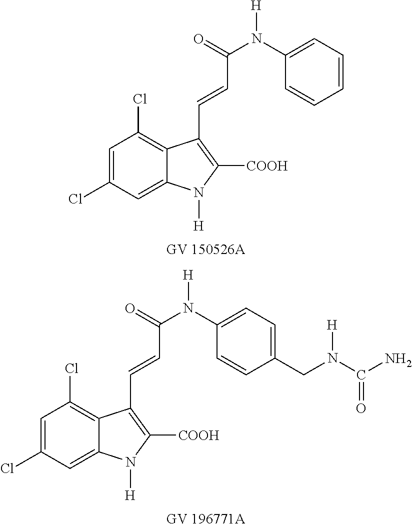 [SMILES: O=C(/C=C/c1c(C(=O)O)[nH]c2cc(Cl)cc(Cl)c12)Nc1ccccc1 ; NC(=O)NCc1ccc(NC(=O)/C=C/c2c(C(=O)O)[nH]c3cc(Cl)cc(Cl)c23)cc1]
or a pharmaceutically acceptable salt or prodrug thereof.

40. The method of claim 35, wherein the NMDAR antagonist is kynurenic acid or a derivative thereof.

41. The method of claim 40, wherein the NMDAR antagonist is selected from the group consisting of: 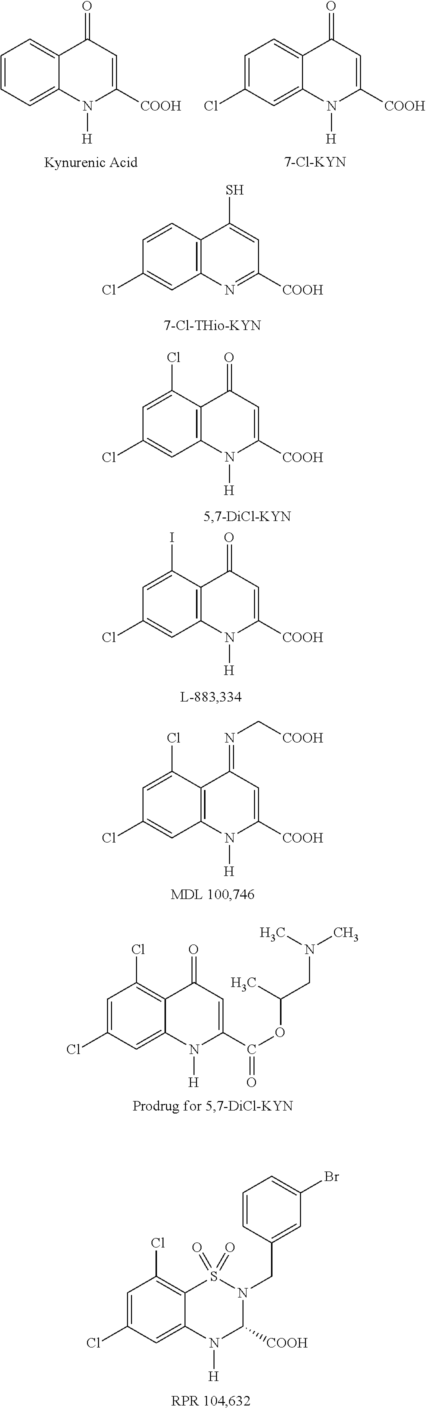 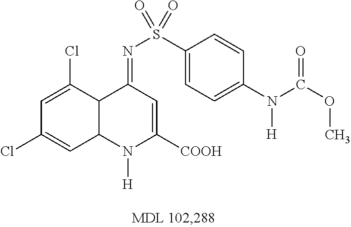 [SMILES: COC(=O)Nc1ccc(S(=O)(=O)/N=C2\C=C(C(=O)O)NC3C=C(Cl)C=C(Cl)C23)cc1]
or a pharmaceutically acceptable salt or prodrug thereof.

42. The method of claim 35, wherein the NMDAR antagonist is a 4-hydroxyquinoline or a derivative thereof.

43. The method of claim 42, wherein the NMDAR antagonist is selected from the group consisting of: 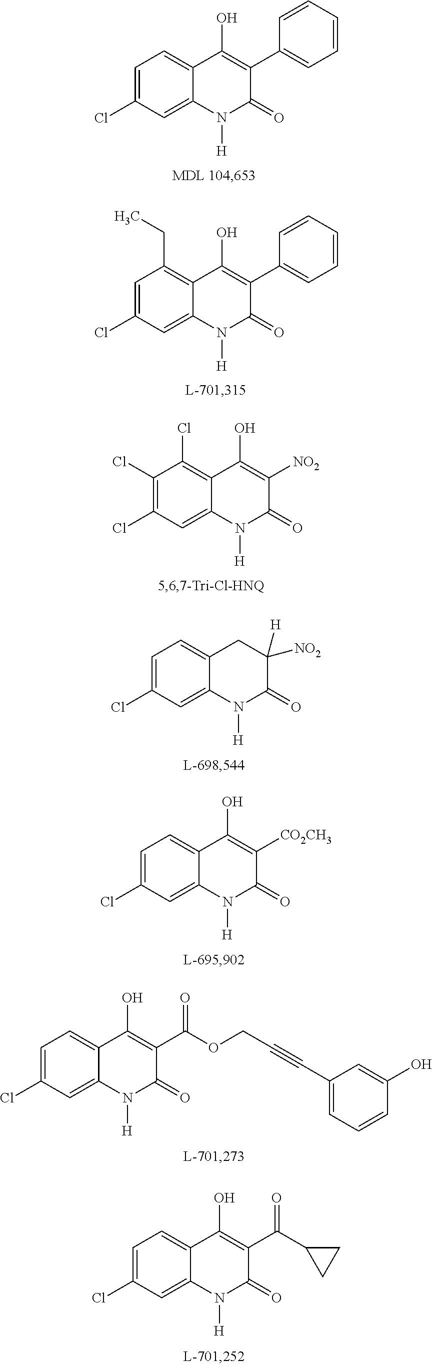 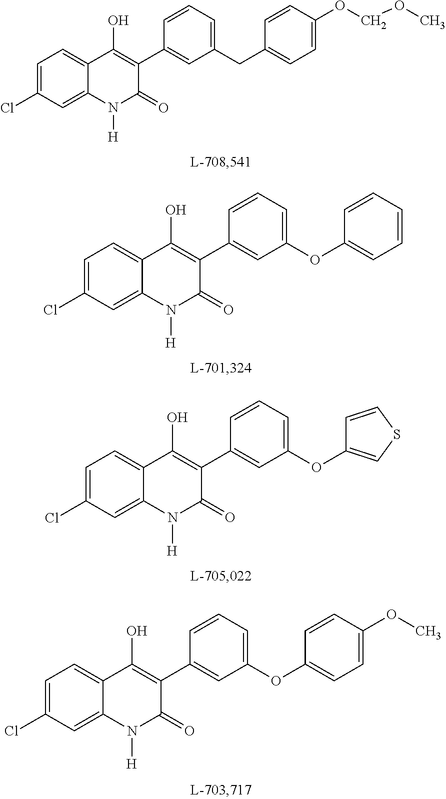 [SMILES: COCOc1ccc(Cc2cccc(-c3c(O)c4ccc(Cl)cc4[nH]c3=O)c2)cc1 ; O=c1[nH]c2cc(Cl)ccc2c(O)c1-c1cccc(Oc2ccccc2)c1 ; O=c1[nH]c2cc(Cl)ccc2c(O)c1-c1cccc(Oc2ccsc2)c1 ; COc1ccc(Oc2cccc(-c3c(O)c4ccc(Cl)cc4[nH]c3=O)c2)cc1]
or a pharmaceutically acceptable salt or prodrug thereof.

44. The method of claim 35, wherein the NMDAR antagonist is a quinoxaline-2,3-dione or a derivative thereof.

45. The method of claim 44, wherein the NMDAR antagonist is selected from the group consisting of: 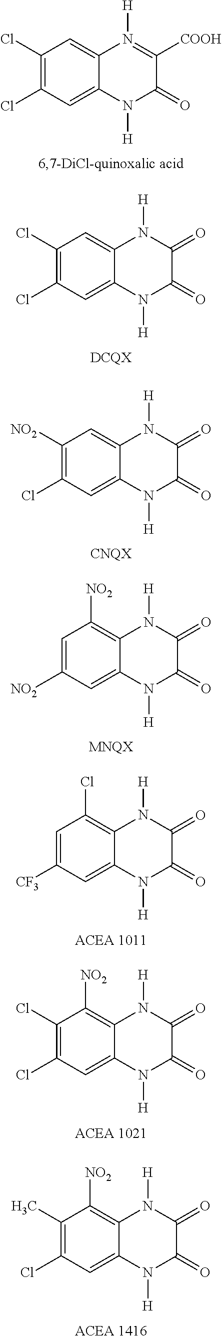 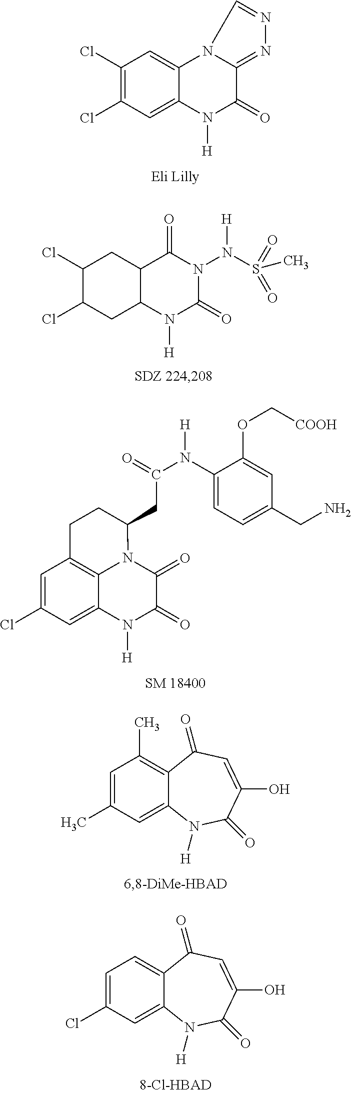
or a pharmaceutically acceptable salt or prodrug thereof.

46. The method of claim 35, wherein the NMDAR antagonist is a tricyclic antagonist.

47. The method of claim 46, wherein the NMDAR antagonist is selected from the group consisting of: 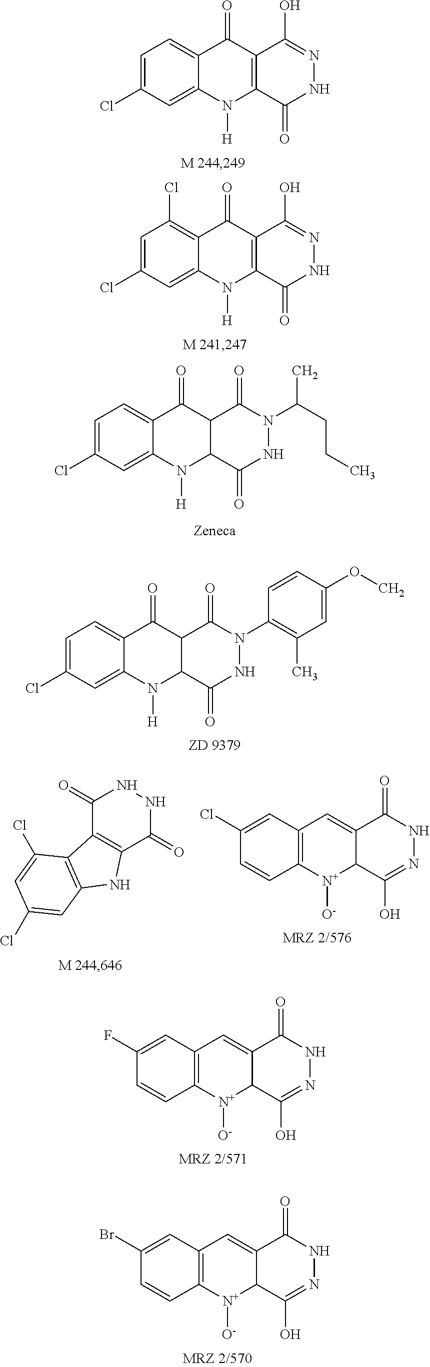
or a pharmaceutically acceptable salt or prodrug thereof.
